(19) United States
(12) Patent Application Publication
(10) Pub. No.: US 2012/0207701 A1
(43) Pub. Date: Aug. 16, 2012

(54) BI-MODAL HYALURONATE

(76) Inventors: Rolf Bergman, Uppsala (SE); Maria Lundqvist, Storvreta (SE); Stig Mannberg, Uppsala (SE); Björn Lundgren, Uppsala (SE); Robert Shimizu, Laguna Niguel, CA (US)

(21) Appl. No.: 13/396,328

(22) Filed: Feb. 14, 2012

Related U.S. Application Data
(63) Continuation of application No. 11/470,988, filed on Sep. 7, 2006, now abandoned.
(60) Provisional application No. 60/715,016, filed on Sep. 7, 2005.
(60) Provisional application No. 60/715,926, filed on Sep. 8, 2005.

(51) Int. Cl. A61K 31/728; A61P 27/02; A61K 31/737; A61K 31/785; A61K 31/717; A61K 31/731

(52) U.S. Cl. 424/78.04; 514/54; 514/57

(57) ABSTRACT

Formulations and methods for protecting cell layers and tissues exposed to trauma are disclosed. Different molecular mass fractions of the same polymer can be used to better control the physical and chemical properties of the formulation. In this manner the benefits of both fractions, and not just the benefits of the average of the fractions, can be seen in the formulation.

### PRIORITY

This application claims priority to U.S. Provisional Patent Application No. 60/715,016, filed Sep. 7, 2005 and U.S. Provisional Patent Application No. 60/715,926, filed Sep. 8, 2005, both of which are incorporated herein in their entirety.

### FIELD OF THE INVENTION

The present invention relates to compositions and methods useful during cosmetic and surgical procedures. More specifically, this invention relates to compositions and methods involving polymers having differing molecular mass for protecting cell layers and tissues exposed to trauma, for example the trauma that is experienced during surgery.

### BACKGROUND

When the natural lens of the eye becomes hazy or clouded, surgery is often performed to remove the impaired lens. During such surgery, the lens can be broken into a number of different particles and then removed using phacoemulsification. Ophthalmic viscosurgical devices (OVDs) are used to protect intraocular tissues, including the corneal endothelium, and maintain space during the surgical procedures.

The characteristics of OVDs are determined by a number of factors, including the polymer used, the molecular mass of the polymer, and the concentration of the polymer. By way of example, and not of limitation, hyaluronic acid (HA), hydroxyl propyl methyl cellulose (BPMC), chondroitin sulfate (CS), carrageenans and polyacrylamide are all known polymers which have been used to formulate OVDs.

Balazs U.S. Pat. Nos. 4,141,973 and 4,303,672 teach mono-modal formulations of hyaluronate suitable for ophthalmic use, in which the hyaluronate fraction falls within the range of 750K to 1.2 million, or i million to 4.5 million, respectively. Although such formulations are useful, it was later discovered that certain bi-modal formulations with hyaluronate fractions up to 4 million can have superior properties. For example, Francese et al., U.S. Pat. Nos. 6,107,347 and 5,492,936 disclose a bi-modal formulation comprising a first alkali metal and/or alkaline earth metal hyaluronate fraction having a molecular mass in the range of 2 to 4 million, and a second alkali metal and/or alkaline earth metal hyaluronate fraction having a molecular mass of about 200,000 to about 800,000. These three patents, and all other extrinsic materials cited herein, are incorporated by reference.

While the bi-modal formulations in the Francese patents provide significant benefits over the Balazs formulations, Francese et al. nevertheless failed to appreciate how to manipulate the various fractions to achieve combinations of desired Theological properties.

Rheological properties are influenced by the composition, molecular mass and concentration of the polymers. In the past these three factors have dictated that solutions be formulated in one of two different categories: dispersive or cohesive. Dispersive OVDs are low to medium high viscosity formulations that behave like solutions over the range of shear rates utilized. They can be more difficult to deliver due to the less pronounced shear thinning viscosity and due to the high concentrations that are required for space maintenance. Furthermore, their solution-like properties give dispersive OVDs longer duration in the anterior chamber when surgeons use high flow and increased vacuum. These properties may also make it difficult to remove dispersive OVDs at the end of surgery. If a surgeon fails to remove the OVD sufficiently, it can lead to an increased risk of transient increases in intraocular pressure (TOP).

Cohesive OVDs have higher viscosities at low shear rates, and behave more gel-like at higher shear rates. They can be formulated at lower polymer concentrations, and so can be easier to deliver due to a highly shear thinning viscosity while maintaining space during surgery. Additionally, their cohesive properties at medium to high shear rates make it easier for surgeons to remove these OVDs following surgery. However, under high flow and vacuum settings, there is a risk that a surgeon may prematurely eliminate the cohesive OVD from the anterior chamber.

The problem is that neither dispersive nor cohesive formulations are optimal for all aspects of all surgeries. For example, different viscoelastic characteristics are preferred depending on the intended use of the viscoelastic, as well as the surgical technique of the surgeon. In the case of ophthalmic surgery, for example, those surgeons who prefer faster surgeries with higher flow rates tend to prefer an OVD that retains a comparatively high viscosity under high shear rates. That is, such a surgeon would likely prefer a more cohesive viscoelastic. On the other hand, a surgeon who uses lower flow conditions would be more likely to prefer a more dispersive viscoelastic composition. In the past, some ophthalmic surgeons have compensated for this by using two different viscoelastic agents during the same surgery. Clearly this adds the risk of viscoelastic confusion to a surgery, as well as additional cost and administrative burdens. A single composition that satisfies all of the needs of the surgeon would be preferable.

Some researchers have attempted to address the supposed dichotomy by varying the concentration of the polymers. Others have used a mix of different species of polymers to impart the desired rheological properties. However, there continues to be a need for new formulations that combine both dispersive and cohesive properties. By way of example, and not of limitation, such new formulations are needed in protecting ocular or eye cell layers and tissues exposed to trauma, such as that arising from ophthalmic surgical procedures, and also in protecting other tissues exposed to trauma, such as joints. There is additionally a need for compositions useful for dermatological applications that are not harmful to the body, but which can provide intended results.

### SUMMARY OF THE INVENTION

The present invention provides methods and compositions in which a given ophthalmically acceptable formulation achieves desirable dispersive and cohesive properties by including non-negligible fractions of compounds having average molecular mass that differ by at least four million. Bi-modal, tri-modal and higher-modal compositions are all contemplated.

In one aspect of preferred embodiments one or both of the compounds is a salt of hyaluronic acid.

In other aspects of preferred embodiments, the first fraction has an average molecular mass of at least 4.5 million, more preferably at least 7 million, and most preferably at least 10 million. The second fraction preferably has a molecular mass of less than 1.5 million, in some formulations less than 1 million, and in other formulations between 0.75 and 1 million. These and all other ranges set forth herein include their respective endpoints.

In other aspects of preferred embodiments, the first fraction has an average molecular mass of at least 2 million, more preferably at least 5 million, and most preferably at least 10 million. The second fraction preferably has a molecular mass of less than 1.5 million, in some formulations less than 1 million, and in other formulations between 22,000 and 1.5 million. These and all other ranges set forth herein include their respective endpoints.

As described in detail herein, a variety of solutions may be prepared, and their properties adjusted based on molecular mass as well as concentration of the various fractions. The concentration of polymer in solutions prepared according to the present invention typically ranges from about 10 to about 80 mg/ml, or from about 15 to about 60 mg/ml, or from about 20 to about 30 mg/ml. Knowing that the relative concentrations of the individual fractions may be determined based on the desired properties of the solution as well as the molecular mass of the fractions being used, one of ordinary skill in the art will be able to determine appropriate relative concentrations for the individual fractions.

In still other aspects, the important dispersive and cohesive properties of viscoelastics can be controlled by the percentages and concentrations of the fractions. These characteristics will then give the solution different properties dependent on the magnitude of the shear forces in action.

In one aspect of the invention, different molecular mass fractions of the same polymer are utilized. Since these fractions will have the same refractive index, there is no issue of optical clarity. In other aspects of the invention the different molecular mass fractions comprise different polymers.

The ability to modulate the properties of viscoelastic formulations according to the present invention has potential benefits to a variety of medical applications in which viscoelastic products are used. These indications include ophthalmic surgery, orthopedic uses, dermal/cosmetic uses and modulation of wound healing. The manipulation of rheological and other physical characteristics can provide distinct advantages in performing tissue and medical device manipulations in surgery (eg, manipulation of TOLs and control of tissue during surgery), In addition, these properties can be used to modulate viscoelastic removal (in the case of cataract surgery) or improve viscoelastic retention (in the cases of dermal fillers and orthopedic supplements). By way of example, and not of limitation, examples of ophthalmic surgery may include cataract and glaucoma surgery. By way of example, and not of limitation, orthopedic uses may include synovial injections. Further by way of example, and not of limitation, dermal and cosmetic uses may include dermal fillers.

Any polymer used according to the present invention can be derived from any source that is known in the art, providing that the source can provide material having the purity that is required for the specific application to which the present invention is being used (e.g., medical grade). In some embodiments of the invention, combinations of polymers may be utilized. As detailed herein, many different polymers will be suitable so long as they are (a) biocompatible and (b) internally compatible with one another in solution.

### BRIEF DESCRIPTION OF THE DRAWINGS

Many aspects of the invention can be better understood with reference to the following drawings:

FIG. 1 is a graph showing Theological properties of three monomodal solutions of sodium hyaluronate having a mol. mass of 4,300,000, the solutions varying in concentration from 2 mg/ml to 10 mg/ml;

FIG. 2 is a graph showing rheological properties of five monomodal solutions of sodium hyaluronate of varying molecular mass, but constant concentration;

FIG. 3 is a graph demonstrating the ability to construct a solution having substantially any desired apparent molecular mass (Mₐₚₚ) by changing the proportions and the average molecular mass of each fraction;

FIG. 4 is a plot of the molecular mass distribution of a bimodal formulation as determined using GPC chromatography coupled with Low Angle Light Scattering (LALLS) detection;

FIG. 5 is a graph showing the shear rates of the various solutions according to the present invention;

FIG. 6 is a graph showing viscosity as a function of shear rate for four different formulations;

FIG. 7 is a graph showing the zero shear rate and viscosity under flow for two different bi-modal formulations; and

FIG. 8 is a graph showing the zero shear rate and viscosity under flow for two different bi-modal formulations.

### DETAILED DESCRIPTION

Viscoelastic (VE) formulations can be characterized by a number of different physical parameters. The parameters of greatest interest herein are those judged to be important for explaining/predicting the function of the OVD in different steps during eye surgery, including the following:

1. Rheology

2. Molecular mass

3. Concentration

4. Osmolality

5. Dynamic contact angle

6. Distribution of molecular masses

1. Rheology

Rheology is the science of deformation and flow of matter. A number of important parameters for the VE solutions can be revealed by performing various tests using a cone-plate rheometer.

Shear Viscosity Test. In one such test a VE-solution is put on a plate, and a cone having a defined geometry (diameter and cone angle) is lowered down into the solution, but stilt providing a specified gap between the cone and the plate. Depending on what features will be investigated, a torque (shear stress) is then appliedto the cone and the resulting strain is recorded. The torque can be applied using either a constant (rotation) or oscillating motion. Depending on the proportion of viscous and elastic properties of the solution being tested, the applied shear stress will partly be lost as fictional heat or partly stored.

To design VE solutions to meet the different flows and shear stresses that will occur during eye surgery, it is important to have knowledge about its resistance to flow at different shear rates. That response is found in a viscosity versus shear rate curve. These measurements are performed in a rheometer with, for example, a coneplate arrangement. The measurements of the viscosity are performed at several different but constant shear rates (constant angular velocity of the cone relative to the plate). From the resulting curves [log(viscosity) vs log(shear rate)] the zero shear viscosity (ZS) is extrapolated from the plateau region where the viscosity no longer changes at lower shear rates. To achieve reliable results the rheometer must be able to monitor viscosities at shear rates below 1/1000−1.

There are three points in the shear viscosity curve that can be used as a rough estimate for the shape of the curve. The viscosity at higher shear rates, 1000−1 is considered to be roughly equivalent to that mostly associated with injection and removal of the VE solution. The viscosity at shear rate 1−1 is generally considered to be where movement of surgical tools in the eye and unfolding of the IDLs are generally considered to take place. The zero shear viscosity, extrapolated from the flat part of the shear viscosity curve at lower shear rates, shows the viscosity of the solution at rest.

To confirm that the intended measurements will be performed within the linear viscoelastic range (LVE), researchers usually perform an amplitude sweep before making any rheological measurements. Within this range all imposed deformations should be reversibly recovered, except where frictional heat is lost and the resistance (modulus for shear viscosity) should be constant.

Oscillation Test. In contrast with the “shear viscosity” test performed under constant rotation, there is another useful test based upon oscillation. In the oscillation test the rheometer is programmed to oscillate the measuring cone at a constant frequency, but with increasing amplitude until the modulus of elasticity at shear G′ and the modulus of viscosity at shear G″ show that the “structure” in the VE solutions is broken. The elastic modulus G′ usually first signals that the structure in the solution is broken and the point where this starts to happen is recorded. Both the value of the modulus before break and the degree of deformation at that point are recorded.

Thus the amplitude sweep gives information about the “net work strength” G′, in the LVE, denoted A(Pa), the degree of deformation at break of solution structure, A(%), and the difference, G′−G″, between the modules of elasticity and viscosity at shear.

Frequency Sweep. Frequency sweeps are performed in the LVE region at a constant and “safe” amplitude, determined in the amplitude sweep. The oscillating frequency for the rheometer cone typically starts at a frequency below 0.01 Hz and goes up to 100 Hz. The main interest is focused on the cross over between the modules of elasticity and viscosity at shear. The frequency at cross over (omega) is correlated to the elasticity of the solution and thus also to the molecular mass of the polymers. At this frequency the modules are equal (G′=G″). A low value of (.omega..sub.co) indicates that the solution is more elastic than that showing a cross over frequency at a higher value.

2. Molecular Mass

The mass average relative molecular mass (M^{rm}) of a test solution can be determined from low angular laser light scattering (LALLS) experiments. The light scattering intensity at a low scattering angle (6 degree) is recorded for a series of different degrees of dilution for each formulation. One of ordinary skill in the art will realize that, depending on the equipment and testing protocol, in other testing scenarios the scattering angle may vary from 6 degree. By way of example, and not of limitation, the angle may vary from 3-8 degree. A least square Fit to the values is extrapolated to zero concentration from which the mass average molecular mass is calculated.

By definition, the mass average relative molecular mass M.sub.rm for a typical, mono-modal solution, is expressed as M rm=n i .times. M i 2 n i .times. M i (1) ##EQU1##

where n.sub.i is the number of molecules with mass M.sub.i.

If (1) is applied to a hi-modal or multi-modal formulation to calculate the M.sub.rm, which hereafter will be noted as M.sub.app, to show that it is the mass average molecular mass of a multi-modal formulation. M.sub.app can be expressed as M app=X i .times. M rm .function. (i) 2 X i .times. M rm .function. (i) (2) ##EQU2##

where M.sub.rm(i) now correspond to the distribution averages M.sub.rm of the i.sup.th HA-substance lot in proportions X.sub.i bi/multi-modal formulation.

3. Concentration

Concentration of the polymer in the viscoelastic solution may be determined by any means known in the art. For example, in the case of HA, concentration is preferably determined indirectly, by treating a sample with concentrated sulphuric acid. Under those conditions, sodium hyaluronate is converted into glucuronic acid, which gives a color with carbazole. The intensity of the color is recorded at 530 nm with a spectrophotometer, and the concentration of sodium hyaluronate can be calculated from the intensity.

4. Osmolaity

Osmolality can be calculated from measurements of the freezing point depression. This result is obtained because the value for osmolality has little or nothing to do with the concentration of the polymers. For example, when HA is examined, the result depends mostly on the ions present in the buffer solution, and the number of counter ions (Na.sup.+) carried by the carboxylic groups on the HA. By determining freezing point depression, one can therefore estimate osmolality and then the amount of HA present. All investigated formulations in our study were shown to have osmolalities between (200-400) mOsm/kg, which is the interval recommended in the ISO 15978:2001, the OVD standard.

5. Dynamic Contact Angle

Contact angles on cleaned hydrophilic microscope glass slides were measured as a function of time. A drop of HA solution is put onto the glass slide, and the drop is analyzed by a digital camera and computer software. The computer performs a best fit to the left and right contact angles of the drop, and calculates their mean value.

The end points of this dynamic process were taken as the value of the contact angle at 90 seconds, (.theta..sub.90), and the slope defined as the difference between the contact angle at (.theta..sub.90−.theta..sub.190)/(90−190). This specific time interval was chosen due to a high signal to noise ratio for all formulations.

6. Distribution of Molecular Masses

This ion exchange HPLC method has so far been used to show whether, and to what degree the chosen HA components have overlapping distributions, However, as one of ordinary skill in the art will know, either HPLC and GPC coupled with LALLS may alternatively be used.

While it is preferred that the final solutions are relatively homogeneous, the preparation of the polymer solutions can be done by any means that is known in the art. By way of example, and not of limitation, samples according to the present invention can be prepared in accordance with the following method.

First, the amount of different polymers required based on molecular mass and final solution properties is calculated. The polymers are then placed in an empty vessel to which a buffer solution is also added. This solution is mixed for several hours until it is homogeneous. The solution is then filled into the primary package (i.e., glass cylinders or syringes) and sterilized. One method of sterilization is steam sterilization.

Depending on the application to which the present invention will be used, bimodal or multi-modal solutions prepared in accordance with the above method can be further treated by means known in the art to remove any air bubbles. By way of example, and not of limitation, the solutions can be centrifuged to remove air bubbles. The solution may alternatively or additionally be sterilized by means known in the art. By way of example, and not of limitation, the solutions can be autoclaved for sterility.

The present ophthalmically acceptable compositions are preferably essentially free of protein and essentially non-pyrogenic. The present compositions preferably include less than about 0.5% by weight of protein based on the total weight of first and second alkali/alkaline earth metal hyaluronates. More preferably, the present compositions have no detectable protein content and no detectable pyrogenicity.

The present compositions preferably include a liquid solvent. In one embodiment of the invention, the solvent is water. The present ophthalmically acceptable compositions are preferably sterile, particular when they are designed for use in a biological environment such as the eye.

The present compositions may also include one or more of the following: a buffer and tonicity component(s). When a buffer is used, it is in an amount effective to control the pH of the composition, and to maintain the pH in a range that is acceptable in the setting in which the present invention is being used. By way of example, when the present invention is used as an OVD in the eye, the pH of the present invention is in the range of from about 6 to about 8, and preferably from about 7 to about 7.5. When a tonicity component (or components) is used, they are preferably used in amounts effective to control the osmolality of the solution so that it is suitable in the setting in which the present invention is being used. By way of example, when the present invention is used as an OVD in the eye, the osmolality of the present invention is in the range of from about 200 to about 400, and preferably from about 250 to about 350 mOsmol/kg.

Inclusion of the buffer and/or the tonicity component(s) is particularly useful when the composition is to be used as part of a surgical procedure. As noted above, when the solution is buffered, the ph is generally between 6.5 and 8, and preferably between 7.0 and 7.5. The tonicity adjustor component(s) act to enhance the compatibility between the composition and the corneal tissue and/or avoid damage to the corneal tissue. The solution is preferably isotonic.

Examples of suitable buffers are well known in the art and include, but are not limited to, acetate buffers, citrate buffers, phosphate buffers, borate buffers and the like (and mixtures thereof). Examples of suitable tonicity agents include, but are not limited to, alkaline and alkaline earth metal salts. By way of example, and not of limitation, sodium chloride and/or potassium chloride can be used.

The present compositions may include one or more other components in amounts effective to provide one or more useful properties and/or benefits to the present composition.

When the present invention is used as an QVD during an ophthalmic surgical procedure, it is particularly suited to protect the human or animal ocular or eye cell layers and tissues which are being exposed to trauma. By way of example, the present invention is suitable for use during ophthalmic surgery when the natural lens is broken into a number of pieces prior to its removal. In one embodiment, the method according to the present invention comprises administering a protective amount of the composition to the ocular or eye cell layers and tissues which are subject to exposure to trauma prior to exposure to the trauma. The compositions of the present invention will adhere to, or at least partially coat, the cell layers and tissues and/or provide protection for the cell layers and/or tissues from the trauma imposed by the surgery.

Uses

One example of a surgical procedure for which the present invention is particularly suited is the method by which the natural lens is removed from an eye. In this embodiment of the present invention, a protective amount of a solution according to the present invention is introduced, such as by injection, into the eye. Such introduction can be through an incision formed in the eye, and can be made by any means known in the art including, for example, a cannula or the like. The composition preferably adheres to at least a portion of the eye cell layers and tissues, in particular the corneal endothelium, while allowing sufficient space maintenance such that the surgeon can perform the required steps of the procedure. Once a protective amount of the present invention is in place, the surgeon may then remove the natural lens. The removal of the lens can be through any means known in the art including, but not limited to, phacoemulsification and associated irrigation/aspiration procedure. Without an OVD, for example an OVD according to the present invention, the portions of the eye in proximity to the natural lens (that are subjected to trauma associated with the phacoemulsification) can be damaged.

When desired by the surgeon, the present invention can be removed from the eye using irrigation/aspiration. The removal is comparatively gentle, and may occur without adversely affecting the remaining portions of the eye.

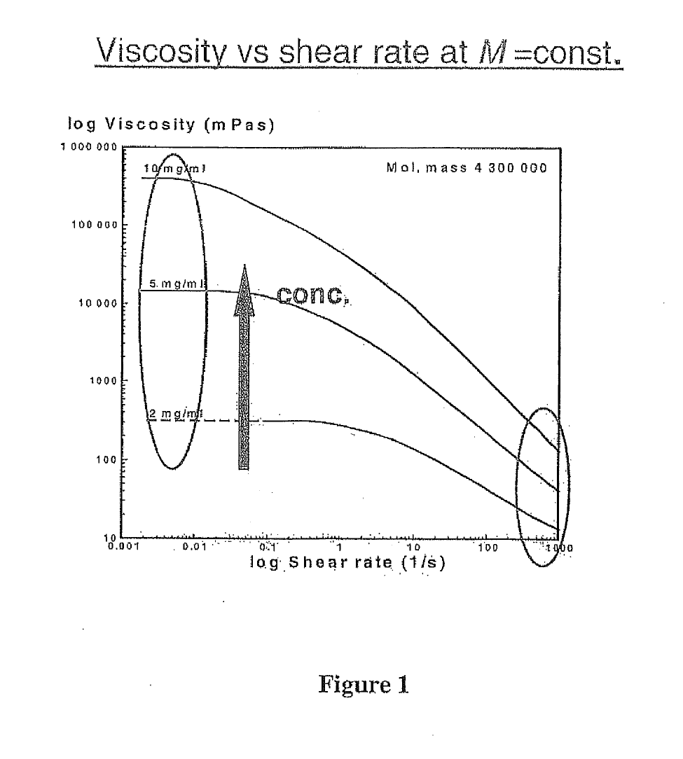

FIG. 1 shows the rheological properties of three monomodal solutions of sodium hyaluronate having a mol. mass of 4,300,000, the solutions varying in concentration from 2 mg/ml to 10 mg/ml. As can be seen, the concentration of the hyaluronate also significantly impacts the rheological properties of the solutions.

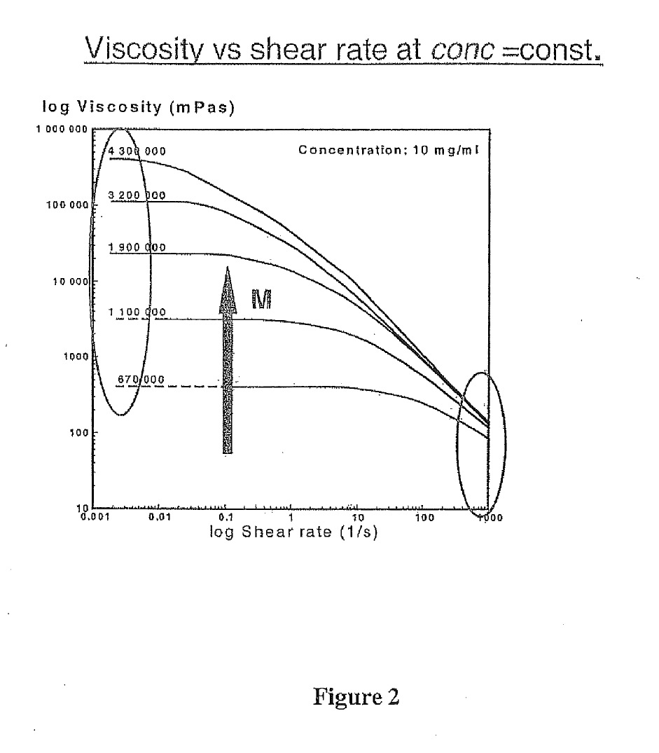

FIG. 2 shows the Theological properties of five monomodal solutions of sodium hyaluronate of varying molecular mass, but constant concentration. As can be seen, the molecular mass of the hyaluronate significantly impacts the rheological properties of the solutions.

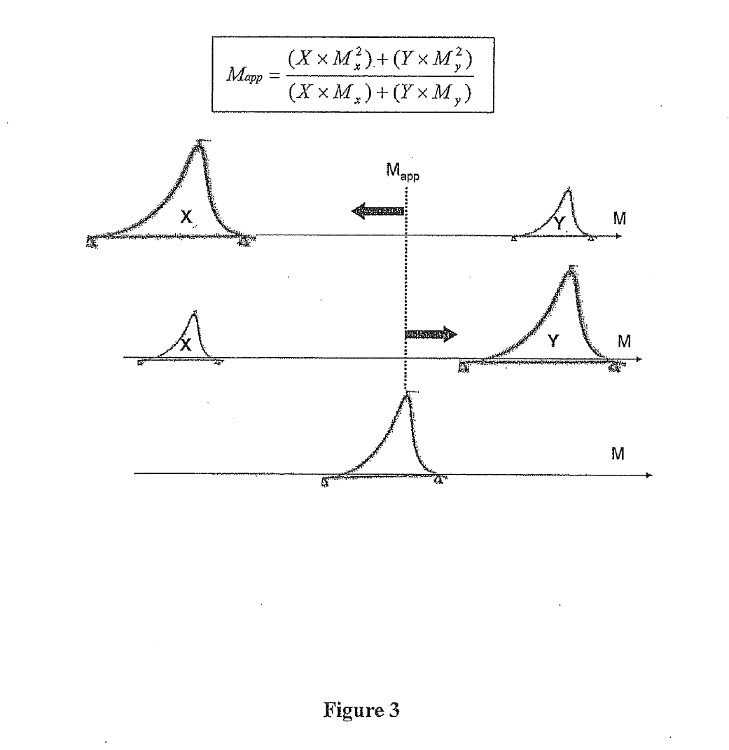

FIG. 3 is a conceptual mapping, showing that it is possible to construct a solution having an apparent molecular mass (M.sub.app) in an infinite number of ways, by changing the proportions and the average molecular mass of each distribution. The rheological properties of the final solution, whether a bi-modal (or multi-modal), will depend on these choices and on the total concentration of the viscoelastic used. Formulations can be modified to broaden the molecular mass distribution and content of HA or other polymer in different areas of the distribution using different polymer fractions.

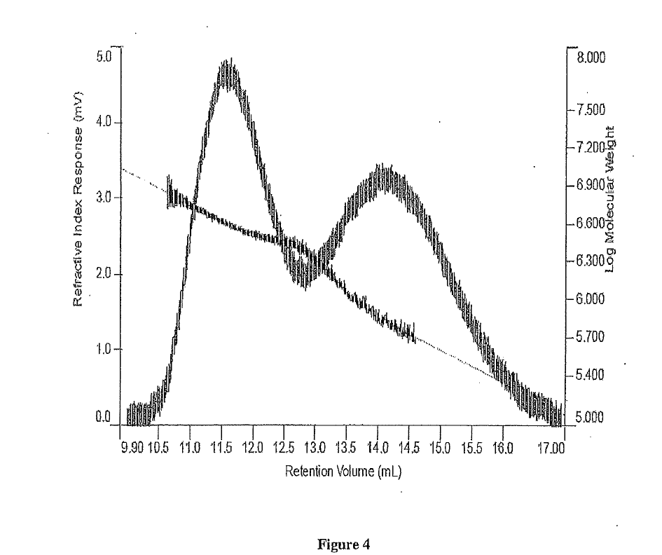

FIG. 4 shows the molecular mass distribution of a bimodal formulation as determined using GPC chromatography coupled with LALLS detection. If the proportion between the high and low molecular mass is changed, it is possible to manipulate/choose the Mapp. Thus, the performance of the solution can be controlled by where the mean of the two populations are and the % composition of each component. By having distinct distributions, it is possible to maximize the benefits of the high and low molecular mass components.

Table 1

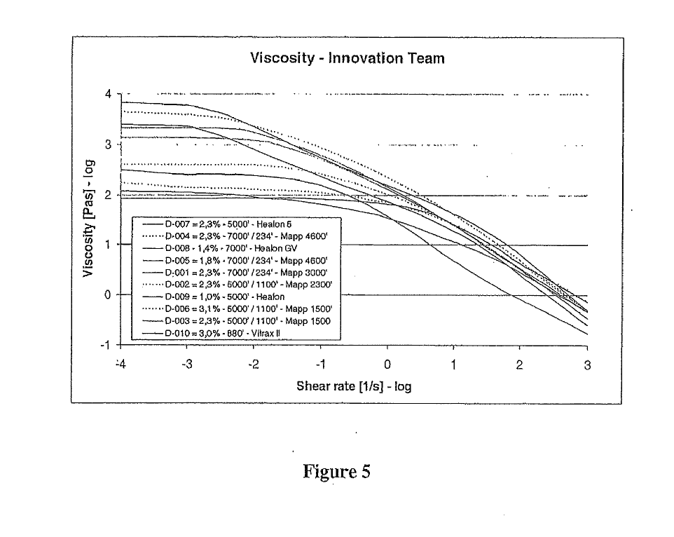

The compositions of the various solutions (M.sub.rm's given are pre-autoclave) noted in FIG. 5 are shown in Table 1: TABLE-US-00001

TABLE 1
Solution Conc. M.sub.rm of High M.sub.rm of Low No. HA MW Fraction
M.sub.rm Fraction M.sub.app D-001 2.3% 7,000,000 234,000
3,000,000 D-002 2.3% 5,000,000 1,100,000 2,300,000 D-003 2.3%
5,000,000 1,100,000 1,500,000 D-004 2.3% 7,000,000 234,000
4,600,000 D-005 1.8% 7,000,000 234,000 4,600,000 D-006 3.1%
5,000,000 1,100,000 1,500,000 D-007 2.3% 5,000,000 N/A N/A
(HEALON 5)) D-008 1.4% 7,000,000 N/A N/A (HEALON GV) D-009
1.0% 5,000,000 N/A N/A (HEALON) D-010 3.0% 880,000 N/A N/A
(VITRAX II)

As can be seen, the control of the concentration and molecular mass of the different fractions significantly affects the performance of the solution. Thus, the performance of the solution can be controlled by where the molecular mass mean of the two populations and the % composition of each component are. Thus, it is possible to maximize the benefits of the high and low molecular mass components.

It is noted that the M.sub.rm of the above compositions are given in pre-autoclave molecular mass, while some of the data is obtained from solutions that have been autoclaved. It is well known that autoclaving has a greater effect on the higher molecular mass polymers. One of ordinary skill in the art will understand that autoclaving has a tendency to break down the polymer to a certain extent, and will be able to calculate or readily determine either (a) the post-autoclaving molecular mass of the solutions as well as their components, or (b) the pre-autoclaving solution(s) which would produce the solutions given herein, sterilized.

While the present invention has been generally described as particularly suited for ophthalmic surgery, one of ordinary skill in the art will realize that the invention may also be utilized in the fields of viscoelastic supplementation (for example, joint administration), cosmetics/plastics (for example, as a dermal filler), and wound healing (for example, as an anti-adhesion agent).

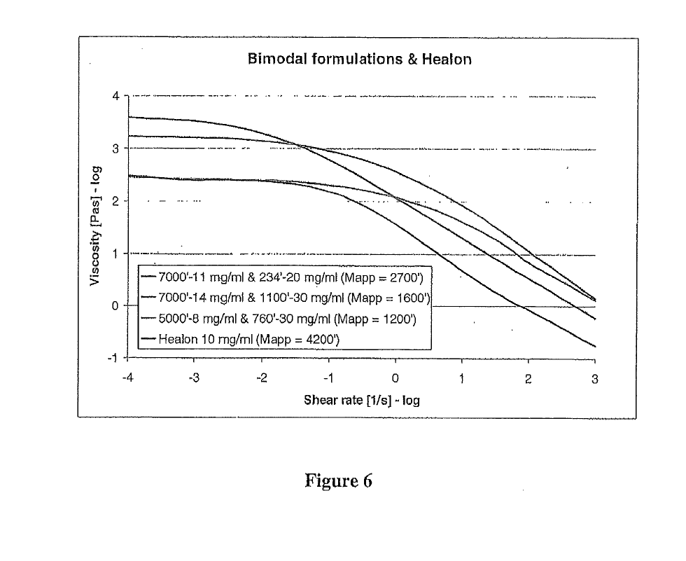

The data shown in FIG. 6 demonstrates that it is possible to control the shear viscosity at different shear rates of importance for different surgical steps of the various solutions according to the present invention. As can be seen, the two bottom curves (labeled 3 and 4) have the same zero shear viscosity, but they are formulated differently. Number 4 is standard mono modal Healon OVD and the bi-modal, number 3, is formulated using a relatively high molecular mass HA, (5 million) at 8 mg/ml, and a relatively low molecular mass (760,000) HA at 30 mg/ml. The total concentration of this solution is thus is 38 mg/ml and the M.sub.app is 1.2 million. These differences are reflected in a higher viscosity on the high shear rate side compared to the standard Healon OVD, which has a concentration of only 10 mg/ml. Both of these formulations have the same space maintaining capability (zero shear viscosity) but with very different flow properties (viscosities at higher shear rates).

Curve number 2 has a higher molecular mass of the high component (7 million, 14 mg/ml) and the “low” mass (1.1 million, 30 mg/ml) is slightly higher than that in the “red curve”. This leads to a slightly higher M.sub.app, (1.6 million) which together with the total concentration 44 mg/ml is reflected on the “low” shear side by a higher shear viscosity in comparison to curve number 3. It should be noted that curve number 2 and curve number 3 start at very different zero shear viscosities However, due to their similar concentrations, they end up with similar viscosities on the high shear rate side.

Curve number 1, a bi-modal curve having in this comparison the highest M.sub.app, of the bi-modals and a total concentration of 31 mg/ml. The total concentration of HA gives this formulation flow properties on the high shear rate side that is in between the Healon OVD (10 mg/ml) and for example the formulation (44 mg/ml) shown with curve number 2. On the low shear rate side where the viscosity is dependent on the concentration multiplied with molecular mass, the decrease in viscosity is steeper due firstly to the high M.sub.app, and secondly to the lower total concentration of HA compared to that in curve number 2.

As is demonstrated by the data presented in the above table the entire shear viscosity curve can be controlled to meet the clinical needs by changing (a) the total concentration of HA, (b) the apparent Molecular mass M.sub.app, and (c) the proportions of the different distributions of HA substances with different M.sub.rm.

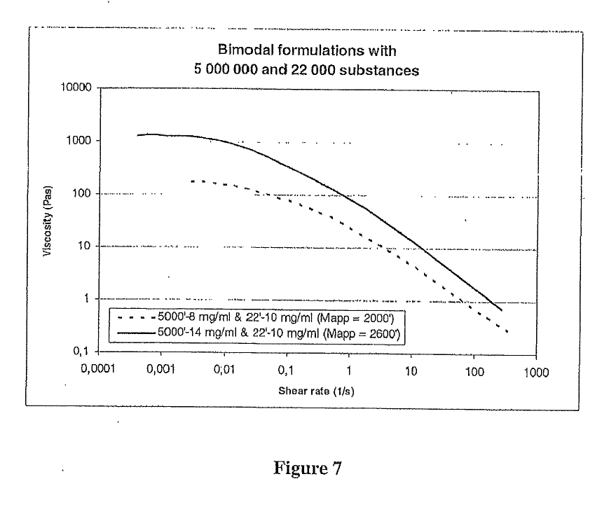

FIG. 7 shows two bi-modal formulations made of a comparatively high molecular mass HA and a very low M.sub.rm=22 000. While these solutions have not too different M.sub.app's, their zero shear viscosities are quite different also due to higher total HA concentration of the upper, solid-line curve.

One of the benefits of the present invention is that it is possible to utilize the benefits of both the high and low molecular mass constituents of the composition. That is, the wettability of a viscoelastic formulation as measured by contact angle on a hydrophilic surface may be changed. Table 2 compares 4 solutions. Each has the same concentration of a high molecular mass and low molecular mass fraction. The main difference between the solutions is the molecular mass of the lower mass fraction, and the resulting contact angle. The contact angle associated with the solutions having the lower, lower molecular mass components is lower than the contact angle associated with the higher, lower molecular mass component. Since a lower contact angle is associated with a greater ability to coat/protect tissues, it is expected that solution 1 would provide a greater coating ability.

### TABLE-US-00002

TABLE 2
Amount Amount Total high low Solution Amount comp. comp. High
low Contact No. HA (mg/ml) (mg/ml) (mg/ml) comp. comp. angle 1
18 mg/ml 8 10 5000′ 22′ 23.9 2 18 mg/ml 8 10 5000′ 234′ 27.1
3 18 mg/ml 8 10 5000′ 760′ 30.0 4 18 mg/ml 8 10 5000′ 1100′ 34.6

As may also be seen from the data presented in Table 2, an increase in the difference in molecular mass between the two polymer fractions leads to an accentuation of the desirable properties of the OVDE For example, when comparing solutions 4 and 3, which have a difference of 3.9 million and 4.24 million, respectively, it is possible to see that the higher difference in molecular mass leads to a lower contact angle, and hence greater wetting or coating ability. The same may be said of solution 2, which has a difference in molecular mass of 4.766 and solution 1, which as a difference in molecular mass of almost 5 million. In one embodiment of the present invention, the difference in molecular mass is greater than 4 million. In an alternate embodiment, the difference is greater than 4.5 million, or greater than 5 million. In a still further embodiment, the difference is greater than 6 million.

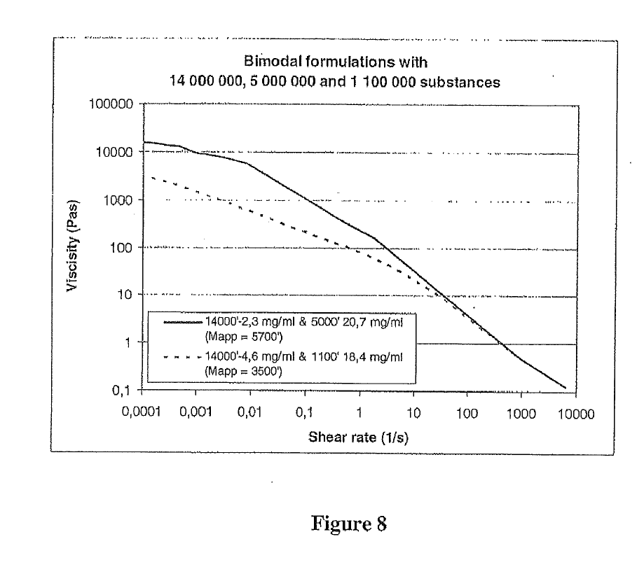

FIG. 8 shows two bi-modal formulations of a super high Mᵣₘ 14,000 with two different “low” HA components. The total concentration is 23 mg/ml in both cases, therefore the viscosity on the high shear side is the same. The difference on the low shear rate side is thus due to the different Mₐₚₚ.

While the foregoing is a complete description of the preferred embodiments of the invention, various alternatives, modifications, and equivalents can be used. Moreover, it will be obvious that certain other modifications can be practiced within the scope of the appended claims. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

1. A formulation comprising:
a first fraction of a first ophthalmically acceptable compound and a second fraction of a second ophthalmically acceptable compound;
wherein the first and second fractions are present in a total concentration of at least 10 mg/ml, and the first and second fractions have average molecular mass that differ by at least 4 million; and
wherein the first and second fractions are adjusted to achieve both dispersive and cohesive properties of the formulation.

2. The formulation of claim 1 wherein the first compound has an average molecular mass of at least 5 million.

3. The formulation of claim 1 wherein the second compound has an average molecular mass of less than 1.5 million.

4. The formulation of claim 1 wherein the first compound has an average molecular mass of at least 5 million and the second compound has an average molecular mass of at least 0.75 million.

5. The formulation of claim 1 wherein the first and second fractions have average molecular mass that differ by at least five million.

6. The formulation of claim 1 wherein the first compound comprises a hyaluronate.

7. The formulation of claim 1 wherein each of the first and second compounds comprises a hyaluronate.

8. The formulation of claim 1 wherein one of the first and second compounds is selected from the group comprising hydroxyl propyl methyl cellulose, carrageenan, chondroitin sulfate and polyacrylamide.

9. The formulation of claim 1, further comprising a third fraction of a third ophthalmically acceptable compound, present in a concentration of at least 5 mg/ml, and wherein the average molecular mass of the third fraction differs from the average molecular mass of at least one of the first and second fractions by at least one million

10. A method of performing surgery using the formulation of claim 1.

11. A formulation comprising:
at least 5 mg/ml each of first and second ophthalmically acceptable, viscoelastic compounds;
the first compound having an average molecular mass of less than 150,000, and the second compound having an average molecular mass of at least 2 million;
the first and second compounds affecting rheology and viscosity based on adjusted desired concentrations and molecular mass of the first and second opthalmically acceptable, viscoelastic compounds.

12. The formulation of claim 11 wherein the second compound has an average molecular mass of at least 3 million.

13. A formulation comprising at least 5 mg/ml each of first and second ophthalmically acceptable, viscoelastic compounds, the first compound having an average molecular mass of less than 2 million, and the second compound having an average molecular mass of at least 5 million; the compounds provide dispersive and cohesive properties by controlling the percentages and concentrations of the compounds.

14. The formulation of claim 13 wherein the first compound has an average molecular mass of less than 400,000.

15. The formulation of claim 13 wherein the first compound has an average molecular mass of less than 300,000.

16. A formulation comprising at least 5 mg/ml each of first and second ophthalmically acceptable, viscoelastic compounds, the concentrations and average molecular mass of the compounds selected such that at a shear rate of 1, the viscosity of the formulation is between 0, 1 and 10 Pas, and at zero shear, the viscosity of the formulation is between 50 and 100,000 Pas.

17. The formulation of claim 16, wherein the formulation has an apparent molecular mass Mₐₚₚ of between at least 1,500,000 and 7,000,000.

18. The formulation of claim 16, wherein the formulation has an apparent molecular mass Mₐₚₚ of between at least 3,000,000 and 7,000,000.

19. The formulation of claim 16, wherein the formulation has an apparent molecular mass Mₐₚₚ of between at least 4,500,000 and 7,000,000.

20. The formulation of claim 16, wherein the first compound is present at a concentration of 1-4% wt/wt.

21. The formulation of claim 16, wherein the first compound is present at a concentration of 2-3% wt/wt.

22. The formulation of claim 16, wherein the formulation is clear to visual inspection.

23. A formulation comprising a first fraction of a first ophthalmically acceptable compound and a second fraction of a second ophthalmically acceptable compound, wherein the first and second fractions are internally compatible with one another in solution and have average molecular masses that differ by at least 4 million, and wherein the difference in average molecular mass accentuates the desirable characteristics of the formulation and provides dispersive and cohesive properties.

24. The formulation according to claim 23, wherein the average molecular masses differ by more than 4.5 million.

25. The formulation according to claim 23, wherein the desirable characteristic is the ability to coat a tissue,

26. A formulation comprising a first fraction of a first ophthalmically acceptable compound and a second fraction of a second ophthalmically acceptable compound, wherein the first and second fractions are internally compatible with one another in solution and have average molecular masses that differ by at least 4 million, and wherein the difference in average molecular mass accentuates either dispersive or cohesive properties by controlling the percentages and concentrations of the fractions.

27. The formulation according to claim 26, wherein the average molecular masses differ by more than 4.5 million.

28. The formulation according to claim 26, wherein the desirable characteristic is the ability to coat a tissue.

29. A method of selecting molecular mass fractions for compounds in a bimodal or multimodal solution and forming the solution comprising:
calculating an amount of a first polymer and an amount of a second polymer based on a desired molecular mass mean and a set of desired final solution properties, wherein the performance of the resulting solution is controlled by the molecular mass mean of the two polymers and the percent composition of the two polymers;
adding the calculated amount of the first and second polymers to a vessel;
adding a buffer solution to the vessel; and
mixing the calculated amount of the first and second polymers and the buffer solution.

30. The method of claim 29, further comprising mixing the calculated amount of the first and second polymers and the buffer solution until the mixture is homogeneous.

31. The method of claim 9, further comprising adding a tonicity component.

32. The method of claim 29, further comprising adding the solution to a primary package.

33. The method of claim 32, wherein the primary package comprises a glass cylinder.

34. The method of claim 32, wherein the primary package comprises a syringe.

35. The method of claim 29, further comprising sterilizing the solution.

36. The method of claim 35, wherein steam sterilization is used.

37. The method of claim 35, wherein an autoclave is used for sterilization.

38. The method of claim 29, further comprising removing air bubbles.

39. The method of claim 38, wherein a centrifuge is used to remove bubbles.

40. The method of claim 29, wherein the set of desired final solution properties includes viscoelastic removal and viscoelastic retention.

41. The method of claim 29, wherein at least one of the set of desired final solution properties is selected based on a relative concentration of the first and second polymer.

42. The method of claim 29, further comprising adding a solvent to the vessel.

43. The method of claim 42, wherein the solvent comprises water.

44. The method of claim 29, further comprising calculating an amount of a third polymer based on a desired molecular mass mean and a set of desired final solution properties.

45. The method of claim 29, further comprising calculating an amount of a multiple additional polymers based on a desired molecular mass mean and a set of desired final solution properties.
